(19) United States
(12) Patent Application Publication
(10) Pub. No.: US 2004/0093751 A1
(43) Pub. Date: May 20, 2004

(54) Apparatus for indicating correct or faulty back posture

(76) Inventor: Randi Henden Tranas, Namsos (NO)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/702,767

(22) Filed: Nov. 7, 2003

Related U.S. Application Data
(63) Continuation of application No. 09/909,833, filed on Jul. 23, 2001, now abandoned.
Application No. 09/909,833 is a continuation-in-part of application No. 09/425,465, filed on Oct. 22, 1999, now Pat. No. 6,279,243.
Application No. 09/425,465 is a continuation of application No. 08/801,304, filed on Feb. 18, 1997, now abandoned.
Application No. 08/801,304 is a continuation of application No. PCT/NO95/00150, filed on Sep. 5, 1995.

(30) Foreign Application Priority Data
Sep. 6, 1994 (NO) 943292

(51) Int. Cl. A61B 5/103

(52) U.S. Cl. 33/512

(57) ABSTRACT

An apparatus for indicating correct or faulty back posture comprises a housing and an indicator body. The housing is supported on the user's body. When the user's back posture or head position is incorrect, the indicator body moves and signals the user of the incorrect posture or position, e.g., through an audible alarm.

This application is a continuation of U.S. patent application Ser. No. 09/909,833, filed Jul. 23, 2001 (abandoned), which is a continuation-in-part of U.S. patent application Ser. No. 09/425,465, filed Oct. 22, 1999 (now U.S. Pat. No. 6,279,243), which is a continuation of U.S. patent application Ser. No. 08/801,304, filed Feb. 18, 1997 (abandoned), which is a § 371 application or a continuation of PCT/NO95/00150, filed Sep. 9, 1995, the entire contents of which are incorporated herein by reference in a manner consistent with the invention and text of this application.

### BACKGROUND OF THE INVENTION

I. Field of the Invention

The present invention relates to a device for placing on a person's head to indicate correct or incorrect upright posture during the performance of various activities in a standing or sitting position, comprising a balance element connected to a headband, a hairband, an ear clip or other appropriate means for attachment to a person's head.

II. Description of Related Art

A device of this kind is taught in the applicant's own Norwegian Patent 171,349. This device consists of a balance body which is attached to a U-shaped band, which in turn is attached to a hairband or similar. A spring or similar is located between the legs of the U-band so that the balance body will tip forward when the head is moved too far forward and out of a correct head position. When this happens, a signal will be given to indicate that the user has assumed an incorrect sitting posture.

This device functions extremely well under qualified guidance, but nevertheless is encumbered with certain weaknesses. For example, the device is rather large and lumpy and projects quite high above the user's head. This means that the device could easily be torn off the head if the user knocks into a low door frame or other obstruction.

Further, the device has proven to be somewhat difficult to calibrate and is very sensitive to deviations from the correct calibration. Moreover, it is difficult for the user to perceive when the balance body of the device tips forward, and he or she is therefore dependent on either a mirror or having another person present as an observer.

Moreover, the device will only indicate incorrect head position when the head is tipped too far forward. It will not indicate an incorrect head position when the head is tipped sideways.

### SUMMARY OF THE INVENTION

In order to eliminate these disadvantages it is therefore proposed to design the device as described in the characterizing clause of independent claim 1 below, and also in the subsequent dependent claims.

### BRIEF DESCRIPTION OF THE DRAWINGS

The device will be described in more detail below with reference to the accompanying drawings, wherein:

FIG. 1 illustrates a device where the moveable body is a slide which can move in a channel in the fixed body;

FIG. 2 shows a device where the moveable body is a pin which, when the head position is incorrect, will move out of a recess in the fixed body;

FIGS. 3a and 3b show the movement of the moveable body according to FIG. 2;

FIG. 4 illustrates a device where the moveable body is a ball which will roll along an inclined plane when the head position is incorrect;

FIG. 5 shows a section through the device in FIG. 4 along the line V-V;

FIGS. 6-9 show a moveable body made of a disc which is designed to move in a hole in the fixed body;

FIG. 6 shows the device from above and with the moveable body in a neutral position;

FIG. 7 shows a section along the line VII-VII in FIG. 6;

FIG. 8 shows the device from above with the moveable body displaced from the neutral position;

FIG. 9 shows a section along the line IX-IX in FIG. 8.

FIGS. 10a and 10b show a preferred embodiment of the device seen in section from above and from the side, respectively;

FIGS. 11a and 11b show the device fastened to the attachment means with a hinged bracket seen from above and from the side, respectively; and

FIG. 12 shows the device in FIGS. 10a and 10b attached to the user's head.

### DETAILED DESCRIPTION OF THE PREFERRED EMBODIMENTS

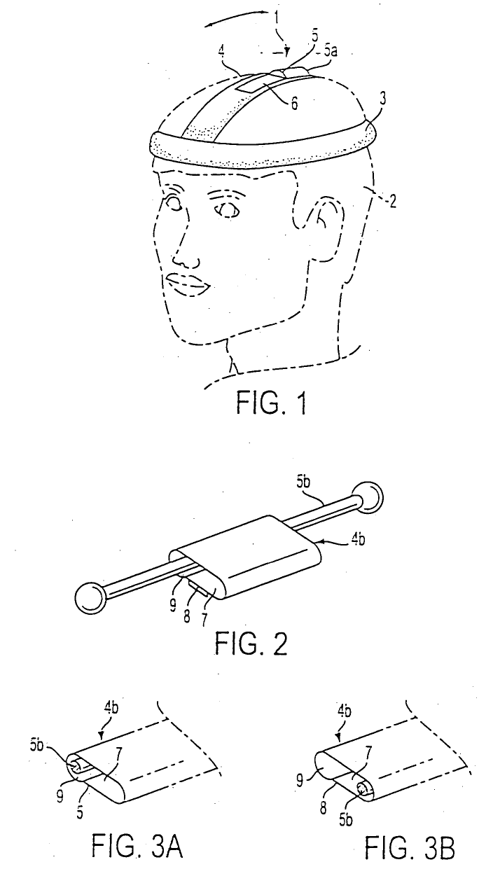

In FIG. 1, a device 1 is shown placed on a person's head 2 with the aid of an attachment means 3, which may be a hairband, a headband or another appropriate means. The device 1 consists of a fixed body 4, which is firmly secured to the attachment means 3, and a moveable body 5, which can move translationally relative to the fixed body 4.

In the exemplary embodiment according to FIG. 1, the moveable body 5 consists of a slide block 5a which is designed to move in a channel 6 in the fixed body 4. When the position of the head is correct, the block 5a will preferably be at the highest point of the head and preferably in the rear end of the channel 6. If the head is bent too far forward, the weight of the block 5a will overcome the frictional forces against the channel 6 and the block 5a will move forward in the channel 6. The materials and design of the block 5a and the fixed body can be chosen so that an audible sound is made when the block 5a strikes the end of the channel 6.

In FIG. 2, a second embodiment of the device is shown. Here, the moveable body 5 consists of a pin 5b and the fixed body consists of a holder 4b equipped with a through-going slot 7 which defines a plane 8 having a recess 9 at the rear end thereof. When the position of the head is correct, the pin 5b is in the recess 9, but when the head is tipped too far forward, it will roll out of the recess 9 and down the plane 8 and preferably make an audible sound when it strikes against the end of the holder 4b. FIGS. 3a and 3b show the position of the pin 5b when the position of the head is correct and incorrect, respectively.

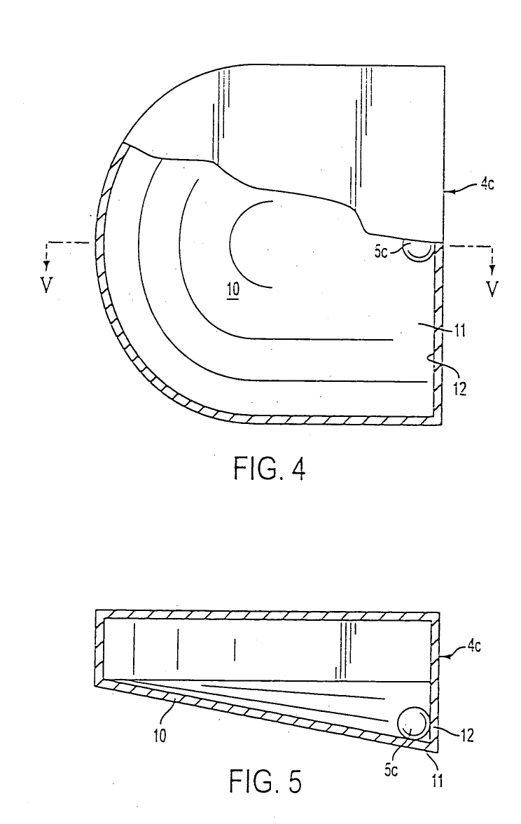

An embodiment is shown in FIG. 4 where the moveable body consists of a ball 5c and the fixed body of a box 4c. The box 4c is equipped with an inclined plane 10 which is preferably curved in such a way that it inclines from the edges of the box and down towards a bottom point 11 at the back edge 12 of the box. In the neutral position, the ball 5c will be at the lowest point 11 of the inclined plane. When the head is tipped too far forward or perhaps to the side, the inclined plane will move past the horizontal position and the lowest point 11 will thereby come to be higher than the inclined plane at the opposite end thereof. The ball will thus roll down the inclined plane and strike against the box wall, preferably making an audible sound in doing so. The box 4c is preferably closed so that the ball 5c is prevented from falling out. The box 4c is attached in an appropriate manner to a headband or the like.

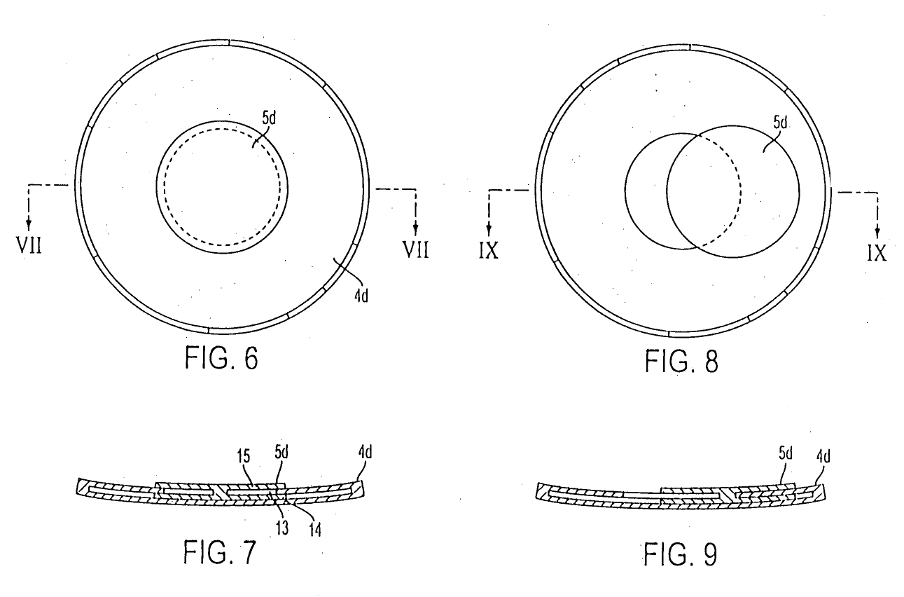

FIGS. 6-9 show a fourth embodiment where the moveable body consists of a disc 5d and the fixed body consists similarly of a disc 4d of greater diameter. As can best be seen in FIG. 7, the disc 4d is double and in one wall has a hole 13 of a smaller diameter than the diameter of the disc 5d. Said disc 5d is also double, but the walls in this case are only connected in the center of the disc. The disc 5d is placed in the hole 13 so that one of the walls 14 of the disc 5d is on the inside of the edge of the hole, whilst the other wall 15 is on the outside. When the position of the head is correct, the disc 5d is centered in the hole 13. On deviation from the correct head position, the disc 5d will shift relative to the hole 13 and clearly indicate that the head position is incorrect. Also in this case, an audible sound will preferably be made when the disc 5d reaches its outermost position. Both the disc 5d and the disc 4d are preferably double curved, so that the device is less sensitive to movement. The curve can be varied according to the desired degree of sensitivity.

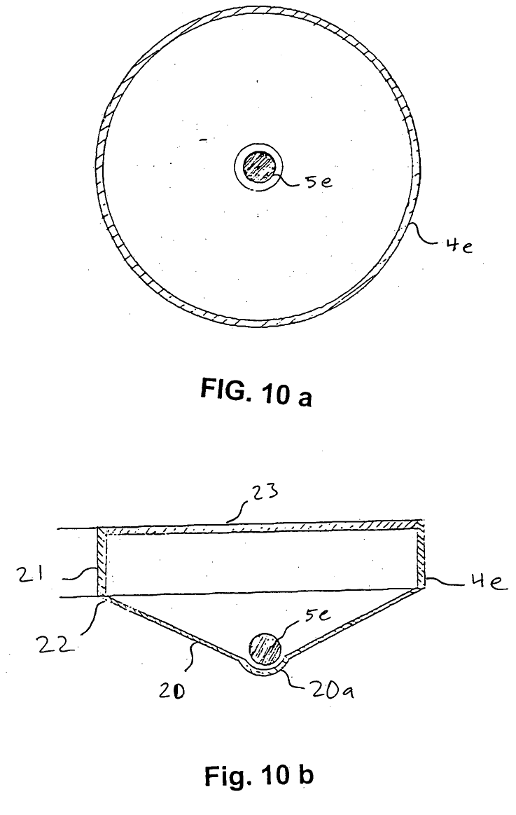

In FIGS. 10a and 10b is shown a preferred embodiment in which the box 4e is fully circular in cross section. The bottom 20 of the box 4e is wedge shaped (or conically shaped) with a lowest point approximately in the center of the box and a circular rim 22 forming the highest point. The lowest point may be situated at an indentation 20a in the bottom 20. The rim 22 joins a circular side wall 21 of the box. The top of the box is closed by a top wall 23, which may have any desired shape, e.g. flat, as shown.

The box 4e may be filled with a fluid with a higher viscosity than air. The fluid may be a liquid. The liquid may be water, alcohol, oil or any other suitable liquid. The purpose of this high viscosity fluid is to slow down the movement of the ball 5e and preventing it from swirling around. Due to the high viscosity of the fluid, the box 4e will have to be tilted somewhat more before the ball 5e starts to roll from the lowest point at the bottom and along the bottom, than what would be the case if the box 4e was filled with air. Consequently, this embodiment allows a little bit more flexibility in defining the correct head position.

The viscosity of the fluid is decisive for the amount of flexibility that is given to the apparatus in defining the correct head position. If, e.g., oil is used, the area defining the correct head position will be larger than if, e.g., water is used.

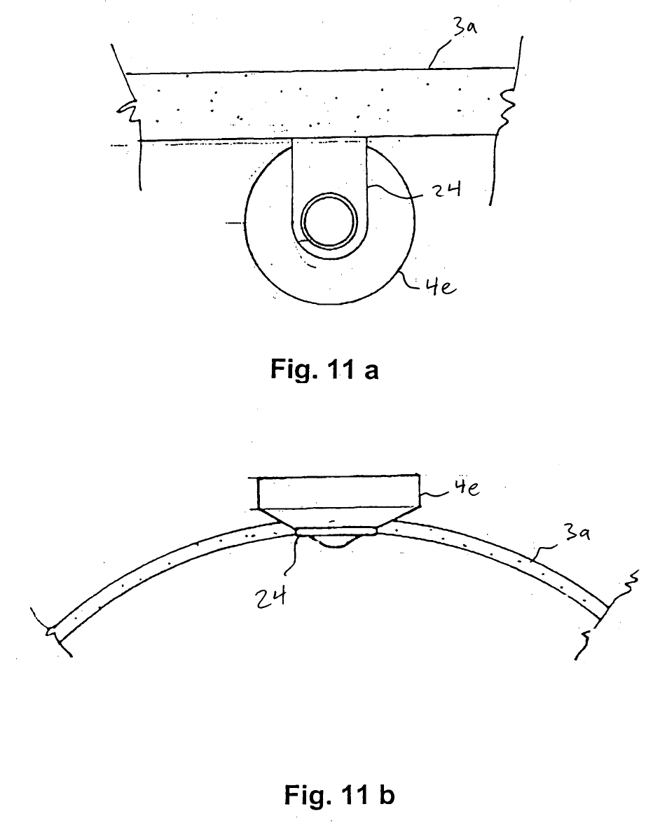

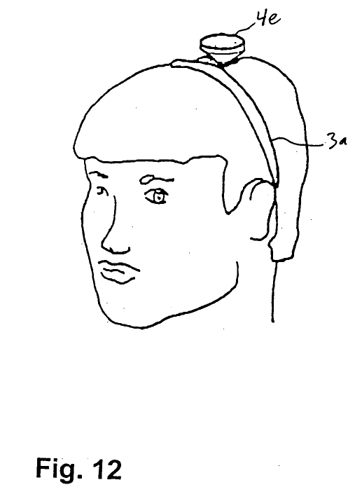

In FIGS. 11a and 11b is shown how the box may be fixed to the head bracket. The box is shown attached to an arm 24, which is shaped as a flat piece. The attachment between the box and the arm 24 may be fixed or detachable, e.g., by a threaded connection.

The arm 24 is preferably hingedly connected to a bracket 3a of the attachment means. The hinge may be a so-called film hinge formed by a thin flexible piece of material. The arm 24 is conveniently situated on the underside of the bracket 3a. When the bracket 3a is placed on the person's head, the arm 24 will be pressed against the bracket 3a. The arm 24 may also be placed on the upper side of the bracket 3a and lie against the bracket 3a by gravity. A snap connection between the arm 24 and the bracket 3a to hold the parts together is also conceivable. It is also conceivable that the connection between the arm 24 and the bracket 3a consist of a hook and loop fastener mechanism (such as Velcro®).

The hinge connection facilitates the packaging and transportation of the apparatus, since this can be laid flat inside a box or the like.

In an embodiment that is not illustrated, the moveable body may consist of a mercury ball or similar which on deviation from the correct head position moves in such a way that it closes an electronic circuit. The electronic circuit may be formed so that it emits an audible sound, e.g., a piping sound, actuates a vibration device, gives a light signal or in another manner indicates that an incorrect head position has been assumed.

The device may also be equipped with a proximity sensor which detects the position of the moveable body and gives a continuous or stepwise signal dependent on the position.

I claim:

1. An indicator device for indicating correct and incorrect head positions of a person's head, comprising:
a base which fits onto the person's head, the base having a shape which adapts to a contour of the person's head;
a housing attached to the base, the housing comprising a top wall, a sloped bottom wall and a side wall, at least a portion of the bottom wall being generally conically shaped, and comprising a first end and a second end, the first end being situated at an indentation formed in the bottom wall and the first end being at a lower position than the second end, the second end being at the perimeter of the bottom wall, the first end being situated at a distance from the perimeter;
an indicator body enclosed and retained in the housing, the indicator body being moveable in a plurality of directions from a start position, the start position of the indicator body corresponding to the correct head position of the person's head and wherein the start position is lower than the perimeter and is located at the first end;
the housing being at least partially filled with and retaining a fluid with a higher viscosity than air; and
wherein when the person's head is deviated from the correct head position to the incorrect head position, the indicator body is deviated in a direction corresponding to a direction from the correct head position to the incorrect head position of the person's head, and the indicator body moves from the start position to one of the plurality of directions and strikes the side wall of the housing causing an audible signal to indicate the incorrect head position.

2. An indicator device according to claim 1, wherein the bottom wall is generally circular, and the first end is situated equidistant from the perimeter of the bottom wall.

3. An indicator device according to claim 1, wherein the first end is situated at an indentation in the bottom wall.

4. An indicator device according to claim 1, wherein the housing is filled with a fluid with a higher viscosity than air.

5. An indicator device according to claim 1, wherein the fluid is a liquid.

6. An indicator device according to claim 5, wherein the fluid comprises a fluid including at least one of: water or alcohol.

7. An indicator device according to claim 5, wherein the fluid comprises oil.

8. An indicator device for indicating correct and incorrect head positions of a person's head, comprising:
a base which fits onto the person's head, the base having a shape which adapts to a contour of the person's head;
a housing attached to the base, the housing comprising a top wall, a sloped bottom wall and a side wall, the bottom wall being generally wedge shaped, and comprising a first end and a second end, the first end being situated at an indentation formed in the bottom wall and the first end being at a lower position than the second end, the second end being at the perimeter of the bottom wall, the first end being situated at a distance from the perimeter;
an indicator body enclosed and retained in the housing, the indicator body being moveable in a plurality of directions from a start position, the start position of the indicator body corresponding to the correct head position of the person's head and wherein the start position is lower than the perimeter and is located at the first end; and
wherein when the person's head is deviated from the correct head position to the incorrect head position, the indicator is deviated in a direction corresponding to a direction from the correct head position to the incorrect head position of the person's head, and the indicator body moves from the start position to one of the plurality of directions and strikes the side wall of the housing causing an audible signal to indicate the incorrect head position;
said housing being at least partially filled with and retaining a fluid with a viscosity greater than air.
